(19) United States
(12) Patent Application Publication
(10) Pub. No.: US 2026/0034054 A1
(43) Pub. Date: Feb. 5, 2026

(54) DEVICES, SYSTEMS, AND METHODS FOR THE TREATMENT OF MALIGNANT NEOPLASM DISORDERS USING CONTROLLED RELEASE DEVICES

(71) Applicant: BIONAUT LABS LTD., Herzliya (IL)

(72) Inventors: Roberta POCEVICUITE, Pasadena, CA (US); Alex KISELYOV, San Diego, CA (US); SueHyun CHO, Torrance, CA (US); Angeliki Maria NIKOLAKOPOULOU, Los Angeles, CA (US); Samantha FIGUEROA, Pasadena, CA (US); Phillip CHEN, Arcadia, CA (US); Michael SHPIGELMACHER, Los Angeles, CA (US); Florent CROS, Los Angeles, CA (US)

(73) Assignee: BIONAUT LABS LTD., Herzliya (IL)

(21) Appl. No.: 19/100,070

(22) PCT Filed: Aug. 7, 2023
(86) PCT No.: PCT/US2023/071781

§ 371 Date: Jan. 30, 2025

Related U.S. Application Data
(60) Provisional application No. 63/396,415, filed on Aug. 9, 2022.

(51) Int. Cl. A61K 9/00; A61K 47/34

(52) U.S. Cl. CPC A61K 9/0024; A61K 47/34

(57) ABSTRACT

Described are devices, systems, and methods for controlled delivery of therapeutic and/or diagnostic agent(s) in a tissue in a subject. In particular, provided are carrier devices in which the agent(s) may be enclosed. One or more carrier devices may be injected or implanted at target loci in a tissue, such as in a malignant neoplasm. The agent(s) may diffuse from the carrier devices in a predetermined and controlled manner. Further provided are methods for developing optimized parameters for intraparenchymal placement of carrier devices and diffusion of the agent(s), to achieve an optimized therapeutic exposure in the target tissue. Further provided are devices and systems for intraparenchymally placing carrier devices at desired loci in a tissue.

### FIELD OF INVENTION

The present disclosure relates to devices, systems, and methods for controlled delivery of a pharmaceutical composition to a subject in need thereof.

### BACKGROUND

Many methods and approaches exist for delivery of a therapeutic agent to the inside of a subject's body: the agent could be ingested, absorbed, and circulated throughout the subject; it could be injected intravenously; delivered by transdermal patch; or any of myriad other options, each of which is useful in various circumstances.

For localized, prolonged delivery of a therapeutic agent to a subcutaneous tissue, options are limited. In some cases, for example, a therapeutic may be delivered locally by feeding an intravenous catheter, but this is highly invasive and expensive. In most cases, a medical professional will perform some form of simple subcutaneous hypodermic injection of an aqueous therapeutic agent solution in the vicinity of a target tissue, such as a malignant neoplasm. This approach, however, of a short quick dose of the therapeutic agent at the target site results in highly uneven therapeutic agent exposure through the target tissue—the section of tissue nearest the injection site will be exposed to a high dose of therapeutic, and more distant sections of tissue may be exposed to a much lower dose. In the context of cancer treatment, this may allow some cells to survive exposure to a lower-than-desired dose of therapeutic agent, which may allow some cancer cells to survive, and may even select for survival and proliferation of drug-resistant cells.

For example, the current standard treatment for malignant neoplasm of the brain or a malignant brain tumor is surgical resection followed by radiation therapy. However, recurrence can occur and is often dealt with using regimens of systemic therapeutic agents typically delivered by an intravenous or oral route. Recurrence of the malignant neoplasm is sometimes local, i.e. it manifests in a region that is finite and, to an certain extent, can be delineated using MRI imaging as a support, for example. The treatment of such regional occurrences can be performed using regional therapy, rather than systemically.

One approach of such a regional therapy is local administration using a GLIADEL® Wafer (carmustine inplant). GLIADEL® Wafers are biodegradable discs infused with the anti-cancer drug (chemotherapeutic agent) carmustine, which is released over several weeks, once placed at the location of interest inside the patient. The GLIADEL® Wafers are placed on the surface of the resected tumor beds in recurrent tumors or after initial resection of the tumor.

Another approach of such a regional therapy is taken by Candel Therapeutics, namely, injection of oncolytic viruses using a syringe and a needle.

These approaches all attempt to distribute a finite amount of a therapeutic agent locally and, while confining the therapeutic agent to a small area of interest rather than the whole body, maximize the exposure of pathological tissues that did not get or could not be removed during surgery, to that therapeutic agent. The dispersion of a therapeutic agent within a mass of tissue near any given site (or locus) is governed by myriads of factors and mechanisms including laws of diffusion, mass transport, interstitial fluid flow, and other local physiological and biological processes that are complex and not easy or possible to control.

For example, oncolytic viruses take part in a complex series of biochemical mechanisms, mediated at first with infection of cells by the virus locally. The first step in this cascade of event starts during and just after the injection itself. Once the viral liquid solution is pushed into the tissue, the first objective is for the virus to infect as many cells as possible. In other words, the virus must move around if it is to infect many cells, which is based on the ability of the virus to infect a cell some distance away from the initial injection site, after a certain amount of time. In the case of GLIADEL® Wafers, the exposure is mostly determined by the ability of the agent (carmustine) to escape from the biodegradable polymeric matrix and diffuse into the surrounding tissue. In both cases, the success of the treatment is, in part, attributable to the ability of the agent to disperse or diffuse evenly and efficiently within a mass of tissue.

Specifically, if left “un-aided” (ex: in the absence of a continuous pressure from a small pump for example), a finite bolus of a therapeutic agent might not distribute efficiently beyond the location of the initial locus as dictated by biophysical (ex: Fick's law of diffusion) and physiological conditions. This is especially true when the liquid contains large biologic molecules (MW>>1,000 D) and cells. By way of example, a crude estimation for a biologic entity as large as an adenovirus purely based on Ficks' law of diffusion leads to rates of 0.2-0.5 mm/day. It is generally accepted that the larger the size/molecular weight or related biophysical parameters of a therapeutic entity, the slower it will move through any given tissue. The more limited the ability to move, the more limited the volume of tissue a particular virus can be expected to affect.

Simple solutions include: (a) increase the agent concentration; (b) deliver more with a specific dosing regimen and frequency; and (c) increase the number of injection/administration sites. While these solutions can work, they all have shortcomings. Concentrated doses of therapeutic agent delivered location can “saturate” the surrounding tissues and result in toxicity and other side effects.

Delivering more can lead to the same effects. Moreover, large quantities of liquid tend to flow down paths of least resistance and escape via natural passageways, such as the sub-arachnoid space or the ventricular system in the case of the brain. A bolus of liquid can easily be washed away in unpredictable fashion, depending on a number of factors linked to the method of administration of the bolus (e.g., speed of injection, depth in the tissue, proximity to vasculature, and so on).

While the delivery of the same amount of material in several loci is a good option, in theory; in practice, as in the case of a syringe injection, administration is imprecise both in the amounts delivered and the accuracy of the targeting of loci itself.

Another approach, once the resection is complete, is to spread a paste that is loaded with the agent. This is an indiscriminate approach that also suffers from the same issues mentioned above.

Thus, there is a need for devices, systems, and methods for the regional therapy of the malignant neoplasm during a regional recurrence or after the initial resection. In particular, there is a need for devices, systems, and methods to assist the spatial distribution, delivery and dispersion of a finite amount of a therapeutic in a tissue, specifically in brain tissue that cannot be effectively resected surgically.

Thus, there is a need for devices, systems, and methods for controlled local delivery of a therapeutic that leads to a more effective way to distribute an agent and target it more efficiently to a given volume of tissue. The devices, systems, and methods of the present disclosure fill this long-felt need for controlled local delivery.

### SUMMARY

Provided herein, in various aspects, are devices, systems, and methods for controlled delivery of a pharmaceutical composition to a subject in need thereof effectively distributes that composition and efficiently targets it to a given volume of tissue

In one aspect, provided herein are carrier devices for intraparenchymal placement or implantation to release a pharmaceutical composition into biological tissue, the device comprising: an exterior shell and an interior, and a matrix comprising the pharmaceutical composition, wherein the matrix is contained within the interior, and wherein the carrier device is configured to release the pharmaceutical composition upon intraparenchymal placement.

In some embodiments of the device, the exterior shell comprises a resorbable or biodegradable material. In such embodiments, the resorbable or biodegradable material may comprise polylactic acid, polyglycolic acid, and/or poly (lactic-co-glycolic acid), and/or combinations thereof. In some embodiments, the exterior shell comprises a hydrogel.

In some embodiments, the carrier device has a maximum dimension of between about 500 μm and about 5 cm. In some embodiments, the carrier device has a substantially cylindrical shape. In some embodiments, the carrier device has a cross-sectional diameter ranging from between about 300 μm to about 5 mm. In some embodiments, the exterior shell has a thickness of between about 5 μm to about 500 μm.

In some embodiments, the matrix comprises a liquid, a powder, a gel, a semisolid, a solid, or a combination thereof.

In some embodiments, the interior is substantially filled with the matrix, the matrix comprising a solid and/or semisolid material in which the therapeutic and/or diagnostic agents are dissolved, suspended, or dispersed. The solid and/or semisolid material may be the same material or a different material as a material of the exterior shell.

In some embodiments, the carrier device has an oblong shape. In such embodiments, the oblong shape may have a major axis dimension and a minor axis dimension, and the major axis dimension may be determined by cutting the carrier device to length. In some embodiments, the carrier device has a shape selected from: a sphere or substantially sphere, a spheroid, a hemisphere, a hemispheroid, a barrel, a corpuscle, a cylinder, a ellipsoid, a hemieillipsoid, a lozenge, a nephroid, a teardrop, or a vermiculate. In some embodiments, the shape is substantially rigid. In some embodiments, the shape is flexible.

In some embodiments, the exterior shell is disposed with one or more holes, each hole passing partly or fully through the exterior shell to the interior. In some embodiments, the holes are disposed in an annular band around an outer circumference of the exterior shell. In some embodiments, the holes are disposed on an isolated region of an outer surface of the exterior shell. In some embodiments, the holes are disposed substantially throughout the entirety of an outer surface of the exterior shell. In such embodiments wherein one or more of the holes pass through the exterior shell, such holes may provide an opening through which the matrix may pass to and from the interior of the carrier device. In some embodiments, the holes have a diameter ranging from between about 100 nm to about 300 μm. In some embodiments, the holes are plugged with opercula, the opercula having a thickness of between about 1 μm to about 300 μm. In some embodiments, the opercula comprising chitosan, zein, and/or polyvinyl alcohol (PVA) or a combination thereof.

In some embodiments, the exterior shell has a circumferential opening for access to and from the interior. In such embodiments, the circumferential opening may be sealed by a temporary stopper.

In some embodiments, the pharmaceutical composition comprises a therapeutic agent, a diagnostic agent, or a combination thereof. In some embodiments, the pharmaceutical composition comprises a plurality of therapeutic agents and/or diagnostic agents. In some embodiments, the pharmaceutical composition comprises a dye or other contrast agent.

In another aspect, provided herein are pharmaceutical composition delivery systems, the system comprising: a plurality of carrier devices described herein. In some embodiments, the system comprises at least 5 carrier devices. In some embodiments, the system comprises at least 10 carrier devices.

In still another aspect, provided herein are therapeutic injection systems, the system comprising: a cannula having a tissue-penetrating end and a payload-receiving end, the payload-receiving end configured to receive one or more carrier devices described herein.

In some embodiments, the payload receiving end is coupled to a feeder, the feeder configured to sequentially feed carrier devices into the payload receiving end of the cannula.

In some embodiments, the tissue-penetrating end is positioned at a selected location within a tissue of a subject. In such embodiments, the tissue may be or may be proximate to at least one malignant neoplasm.

In some embodiments, the system further comprises a robot actuator to position the cannula.

In yet another aspect, provided herein are methods of providing a treatment plan for a subject in need thereof, the method comprising the steps of: (a) determining the spatial dimensions of a tissue or subtissue in need of treatment; (b) determining a optimal dose and time course for the treatment; (c) inputting topological and medical data into a general-purpose computer pre-installed with software configured to determine recommended materials, dimensions, size, dosing, and/or spacing of a pharmaceutical composition delivery system as described herein; (d) the general-purpose computer determining and outputting a treatment plan comprising recommended materials, dimensions, size, dosing, and/or spacing for the pharmaceutical composition delivery system.

In still another aspect, provided herein are methods of treating a disease or condition in a tissue of a subject in need thereof, the method comprising: intraparenchymally positioning one or more carrier devices described herein into the tissue of the subject. In some embodiments of the method, two or more carrier devices are sequentially positioned by: (a) introducing a cannula into a first target location in the tissue, and positioning a first carrier device; (b) introducing the cannula into a second target location in the tissue, and positioning a second carrier device; (c) optionally repeating step (b) (for an nth target location in the tissue and positioning an nth carrier device).

In still another aspect, provided herein are methods of treating a malignant neoplasm in a brain of a subject in need thereof, the method comprising: intraparenchymally positioning one or more carrier devices described herein into brain tissue of the subject. In some embodiments of the method, two or more carrier devices are sequentially positioned by: (a) introducing a cannula into a first target location in the brain tissue, and positioning a first carrier device; (b) introducing the cannula into a second target location in the brain tissue, and positioning a second carrier device; (c) optionally repeating step (b) (for an nth target location in the brain tissue and positioning an nth carrier device).

In some embodiments of the method, the tissue may be a malignant neoplasm.

### BRIEF DESCRIPTION OF THE DRAWINGS

The subject matter disclosed may best be understood by reference to the following detailed description when read with the accompanying drawings in which:

FIG. 1 depicts an illustrative schematic of the therapeutic injection system.

FIG. 2 depicts an illustrative drawing of therapeutic carrier devices positioned in situ within or proximate to a malignant neoplasm in a patient's tissue.

FIG. 3 depicts an illustrative drawing of multiple “trajectories” of therapeutic carrier devices positioned in situ within or proximate to a malignant neoplasm in a patient's tissue. The devices may contain diagnostic and/or therapeutic agents.

FIG. 4 depicts an illustration of a carrier device, from which a therapeutic agent may diffuse at a timing and rate that may be predetermined by a user.

FIG. 5 is an exemplary perspective-view photograph of a carrier device positioned next to a U.S. dime for scale. The carrier device in this photograph has a circumferential opening of about 200 μm in diameter at a distal end, and the surface of the exterior shell is disposed throughout with holes, from which a therapeutic agent may diffuse. This exemplary embodiment is about 1 mm in diameter, with the exterior shell made from poly (lactic-co-glycolic acid) (PLGA).

FIGS. 6A-6B depict exemplary photographic views of carrier devices. FIG. 6A depicts a carrier device, shown here during filling with a “pharmaceutical agent”, having an exterior shell made from poly (vinyl alcohol) (PVA). The agent depicted here is a blue dye placebo. The steel tubing is about 300 μm in diameter. FIG. 6B depicts a carrier, about 1 mm in cross-sectional diameter, having an exterior shell made from PLGA, filled with a blue-dye placebo. The device has a circumferential opening at one end, which is shown here sealed with a temporary stopper.

FIG. 7 depicts a scale photograph of a carrier device made from PLGA before and after coating with PVA.

FIG. 8 depicts an exemplary diagram of a series of manufacturing steps for a carrier device, the device exterior shell is machined, then coated, then filled with a pharmaceutical composition, and then sealed with a temporary stopper (also called a “cork”).

FIG. 9 depicts an exemplary time-series of photographs demonstrating the diffusion profile of a dye from a carrier device placed in an agarose tissue-simulant.

FIG. 10 depicts a schematic of an alternate embodiment of a carrier device.

FIG. 11A depicts the 3-dimensional distribution of a viral particles injected into parenchyma of a model organism using a carrier device according to embodiments of the present invention (left side) and by injection performed using a 33-gage needle (right side). FIG. 11B depicts another view of a distribution of a viral particles injected into parenchyma of a model organism using a carrier device according to embodiments of the present invention (left side) and by injection performed using a 33-gage needle (right side).

FIG. 12 depicts a pictogram comparing a traditional hypodermic injection (upper row) versus the carrier device of the present description (lower row).

FIG. 13 illustrates optimized placement of carrier devices in a tissue targeted for treatment. Based on the diffusion profile of the carrier devices, an optimal placement may be calculated, e.g., by a computer, such that all the tissue receives a dose of therapeutic agent for a desired duration.

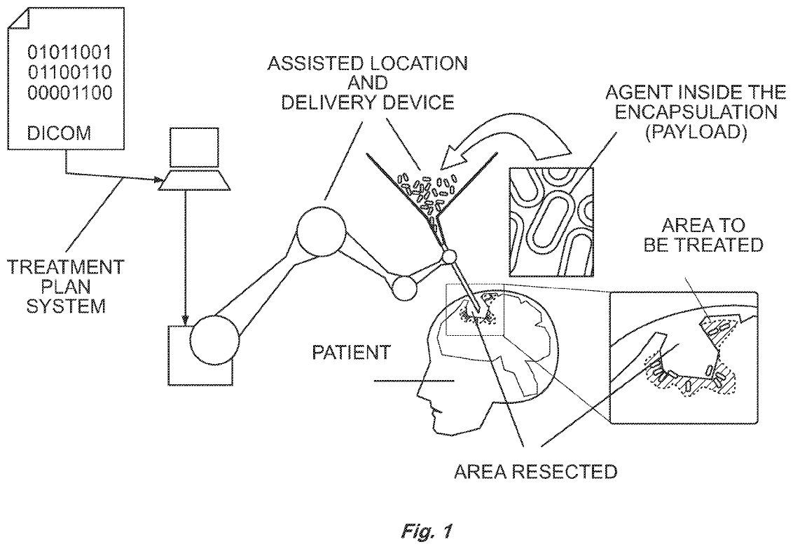

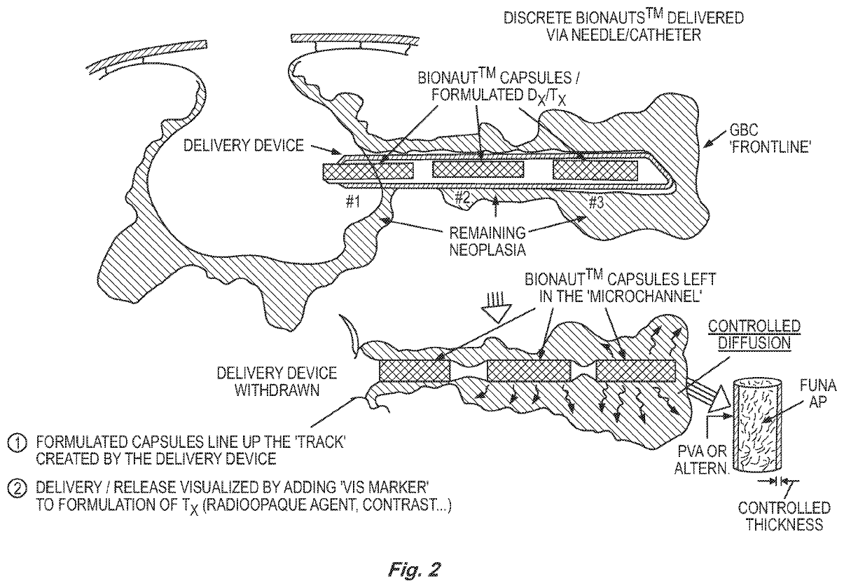

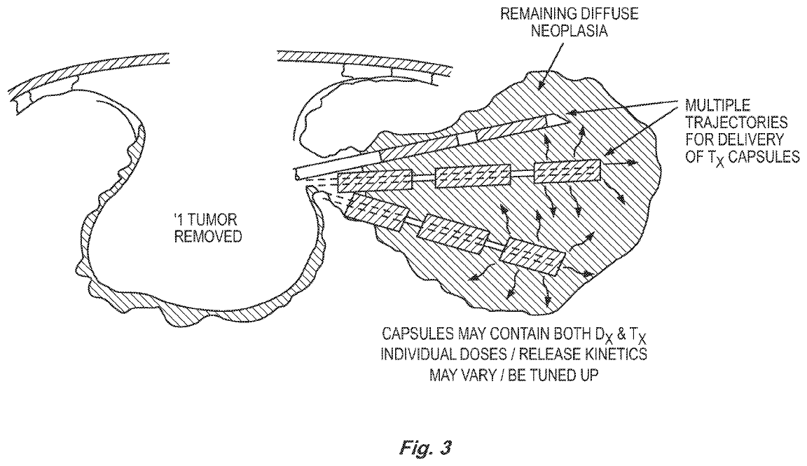

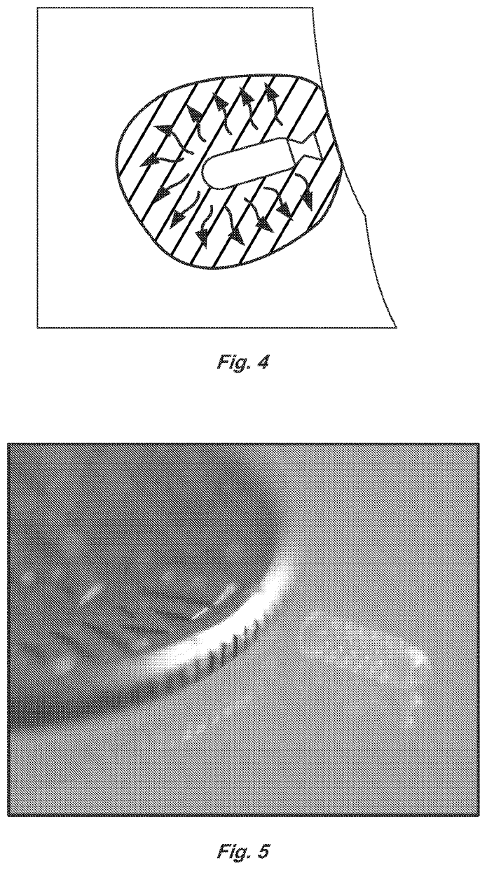

For simplicity and clarity of illustration, elements shown in the figures are not necessarily drawn to scale, and the dimensions of some elements may be exaggerated relative to other elements. In addition, reference numerals may be repeated among the figures to indicate corresponding or analogous elements.

### DETAILED DESCRIPTION

Provided in the present disclosure, in various aspects, are devices, systems, and methods for controlled delivery of a pharmaceutical composition to a subject in need thereof.

The devices, systems, and methods described herein provide for a “controlled diffusion” localized treatment at selected tissues or loci in a mammalian subject. For example, the devices, systems, and methods of the present disclosure may be used to treat multiple neoplastic and/or inflammatory conditions that exhibit ‘diffuse’ nature, i.e., malignant or diseased cells/tissue are intertwined with healthy cells/tissue.

Described here is a small-size carrier device. The device(s) provide a means to temporarily encapsulate a pharmaceutical composition, e.g., comprising a therapeutic agent and/or diagnostic agent, in one or more devices, permitting the pharmaceutical composition to escape in a predictable time, rate, and direction. Once implanted, a single carrier device or multiple carrier devices, each carrier device provides a finite, controlled dose which a controlled-release kinetic and predictable tissue penetration and distribution. A predictable volume of tissue may be thereby exposed to a therapeutic agent. The carrier device may be made from biocompatible and/or resorbable materials, and thus the carrier devices may be completely absorbed by the surrounding tissue. (For purposes of the present disclosure, it should be readily understood that the carrier device may carry any useful agent, and describing that the device carries a “therapeutic agent” or “diagnostic agent” should not be construed as limiting. For example, it will be apparent that the carrier device may carry a dye or other contrast agent.)

Further described herein are means of devising a treatment plan for placement of the carrier devices. A treatment plan system (TPS) may comprise a general-purpose computer preinstalled with specialized software. The methods permit selection of an optimal number of carrier devices at an optimal placement in a target tissue. The size, number, location, solubility, etc. of the carrier devices may be optimized depending on the chosen therapeutic agent, the subject's diagnosis, the physical properties of the target tissue, and the 3-dimensional topology of the target tissue. For example, an aggregate total volume of tissue reached by a therapeutic agent may be as great as 30 cm³ in the brain.

Further described herein are methods of precision placement of carrier devices at the desired target loci in a subject's tissue. An assisted location and delivery system may permit placement of multiple carrier devices.

Thus, in an aspect, provided herein are carrier devices for intraparenchymal placement or implantation to release a pharmaceutical composition into biological tissue, the device comprising: an exterior shell and an interior, and a matrix comprising the pharmaceutical composition, wherein the matrix is contained within the interior, and wherein the carrier device is configured to release the pharmaceutical composition upon intraparenchymal placement.

In some embodiments of the device, the exterior shell comprises a resorbable or biodegradable material. In such embodiments, the resorbable or biodegradable material may comprise polylactic acid, polyglycolic acid, and/or poly (lactic-co-glycolic acid), and/or combinations thereof.

In some embodiments, the exterior shell comprises a hydrogel. In some embodiments, the exterior shell comprises a bioresorbable hydrogel. In some embodiments, the matrix comprises a hydrogel. In some embodiments, the hydrogel is infused the therapeutic and/or diagnostic agents.

In some embodiments, the matrix comprises a desiccated or hydrophilized hydrogel infused with the therapeutic and/or diagnostic agents, to reduce its size, and which may rehydrate and swell upon intraparenchymal placement. For example, once inside the tissue, the desiccated or hydrophilized hydrogel rehydrates from aqueous liquids present in the environment, for example, cerebrospinal fluid (CSF) or interstitial fluids.

In some embodiments, the carrier device is a long continuous soft flexible material, for example, a hydrogel, that has been infused with the therapeutic and/or diagnostic agents. In some embodiments, the carrier device is long and comprises a solid material that has been infused with the therapeutic and/or diagnostic agents and can be provided in predetermined length or cut to size during the delivery in the tissue.

In some embodiments, the carrier device, once inside the tissue, houses the pharmaceutical composition for a few minutes to a few hours prior to letting the pharmaceutical composition escape and come in contact with the tissue. In some embodiments, the carrier device, once inside the tissue, lets the pharmaceutical composition escape at a predetermined rate. In some such embodiments, the predetermined rate of escape may be controlled via physical openings or windows present in the exterior shell.

In some embodiments, the exterior shell comprises a material that can dissolve in aqueous solution, including but not limited to, hypromellose, chitosan, zein, polyvinyl alcohol (PVA) and/or caramel. In some embodiments, the exterior shell is rigid. In other embodiments, the exterior shell is soft and flexible.

In some embodiments, the carrier device has a maximum dimension of between about 500 μm and about 5 cm. In some embodiments, the carrier device has a substantially cylindrical shape. In some embodiments, the carrier device has a cross-sectional diameter ranging from between about 300 μm to about 5 mm. In some embodiments, the exterior shell has a thickness of between about 5 μm to about 500 μm.

In some embodiments, the matrix comprises a liquid, a powder, a gel, a semisolid, a solid, or a combination thereof.

In some embodiments, the interior is substantially filled with the matrix, the matrix comprising a solid and/or semisolid material in which the therapeutic and/or diagnostic agents are dissolved, suspended, or dispersed. In some such embodiments, the solid and/or semisolid material is the same material than that of the exterior shell. In other such embodiments, the solid and/or semisolid material is a different material from that of the exterior shell. In some embodiments, the interior is hollow and substantially filled with the matrix, the matrix comprising a liquid, a powder or a solid material. In some embodiments, the interior is hollow and substantially filled with the matrix, the matrix comprising a liquid, a powder or a solid material. In some embodiments, the external shell is solid and therapeutic and/or diagnostic agents are mixed or dispersed within the interior with no discernable hollow space that delineates the interior from the external shell.

The carrier device can be shaped to be inserted into living tissue, for example it can have rounded corners and an oblong shape. In some embodiments, the carrier device has an oblong shape. In such embodiments, the oblong shape may have a major axis dimension and a minor axis dimension, and the major axis dimension may be determined by cutting the carrier device to length. In some embodiments, the carrier device has a shape selected from: a sphere or substantially sphere, a spheroid, a hemisphere, a hemispheroid, a barrel, a corpuscle, a cylinder, a ellipsoid, a hemieillipsoid, a lozenge, a nephroid, a teardrop, or a vermiculate. In some embodiments, the shape is substantially rigid. In some embodiments, the shape is flexible.

In some embodiments, the exterior shell is disposed with one or more holes, each hole passing partly or fully through the exterior shell to the interior. When the holes do not pass all the way through the exterior shell, they may be described as “dimples”. The dimples may dissolve/resorb more quickly than thick portions of the exterior shell, such that the dimples will open up into through-holes and pharmaceutical composition can diffuse therefrom.

In some embodiments, the holes are disposed in an annular band around an outer circumference of the exterior shell. In some embodiments, the holes are disposed on an isolated region of an outer surface of the exterior shell. In some embodiments, the holes are disposed substantially throughout the entirety of an outer surface of the exterior shell. The holes may be placed or disposed along the exterior shell in any desired “dot” pattern. The holes may be preferentially placed on one side of the carrier device, thus permitting an asymmetric “directed” diffusion of the active agent.

In such embodiments wherein one or more of the holes pass through the exterior shell, such holes may provide an opening through which the matrix may pass to and from the interior of the carrier device. In some embodiments, the holes have a diameter ranging from between about 100 nm to about 300 μm. The holes may have a diameter of about 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, up to about 300 μm.

In some embodiments, the holes are plugged with opercula, the opercula having a thickness of between about 1 μm to about 300 μm. In some embodiments, the opercula comprising chitosan, zein, and/or polyvinyl alcohol (PVA) or a combination thereof. The opercula may have a thickness of about 1 μm, 2 μm, 3 μm, 4 μm, 5 μm, 6 μm, 7 μm, 8 μm, 9 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, up to about 300μm.

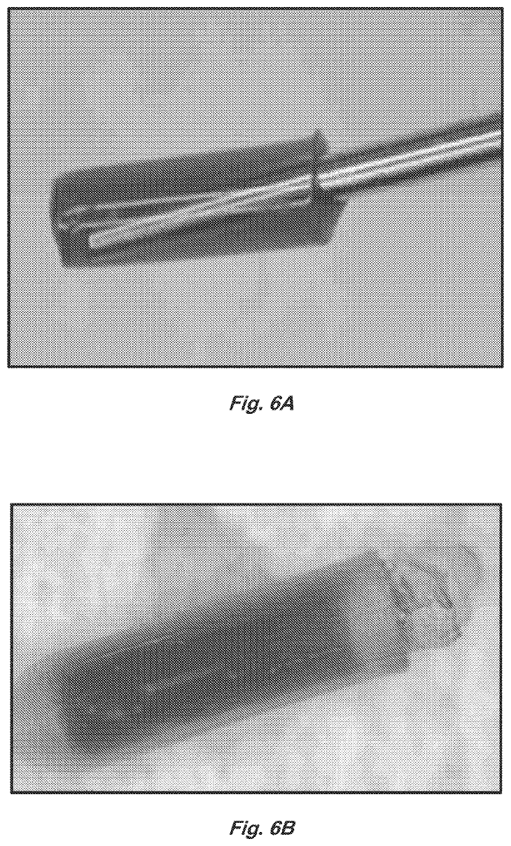

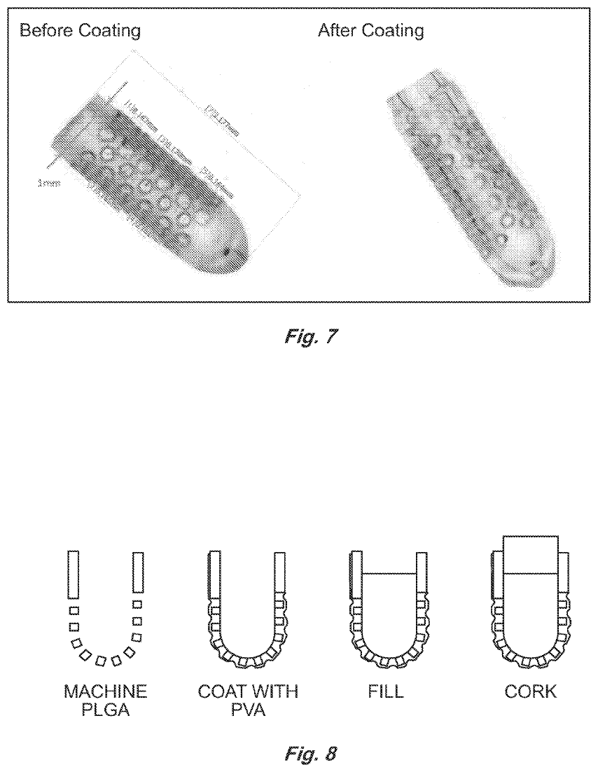

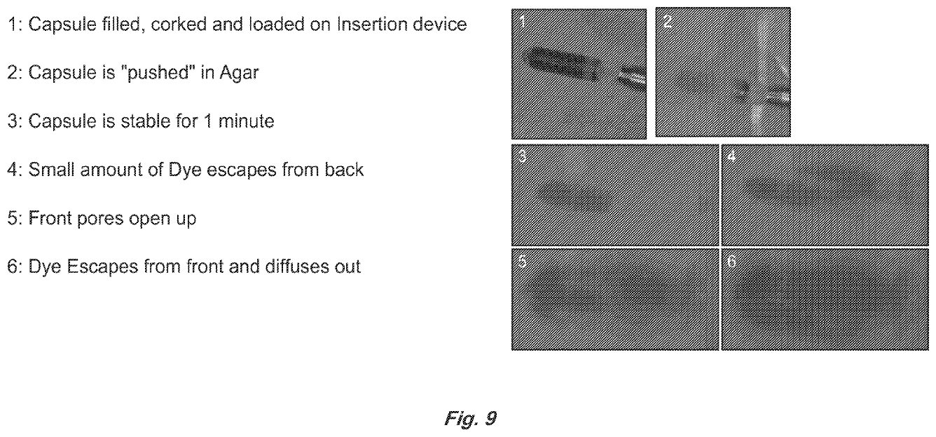

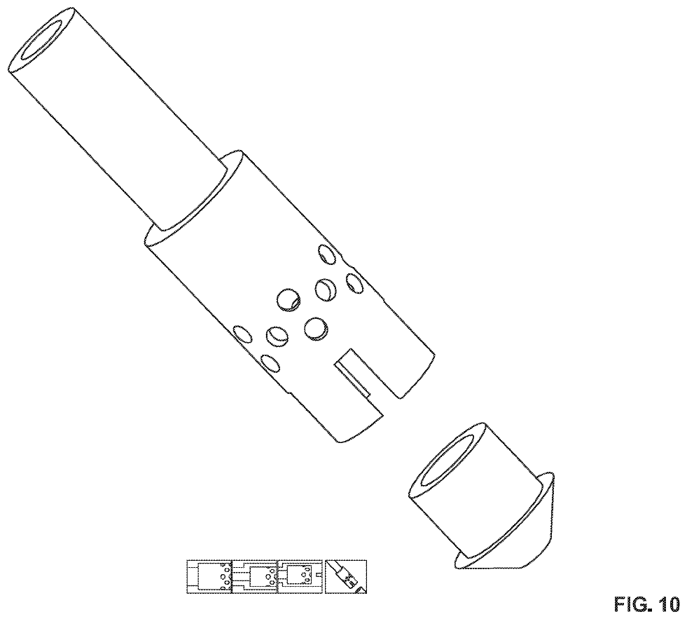

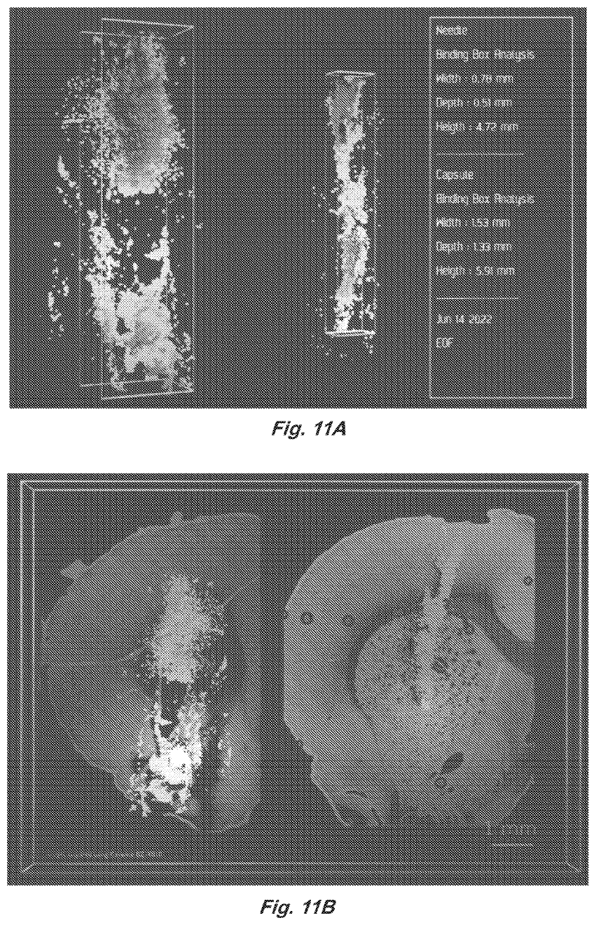

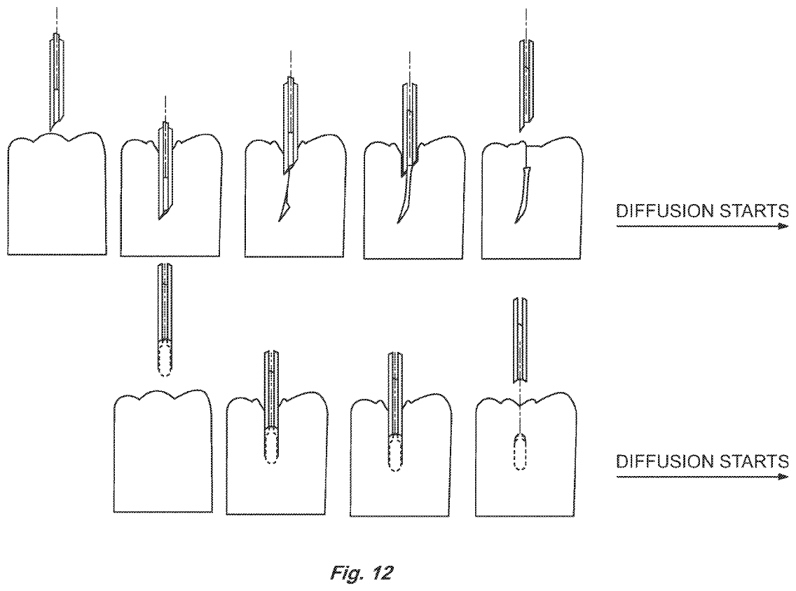

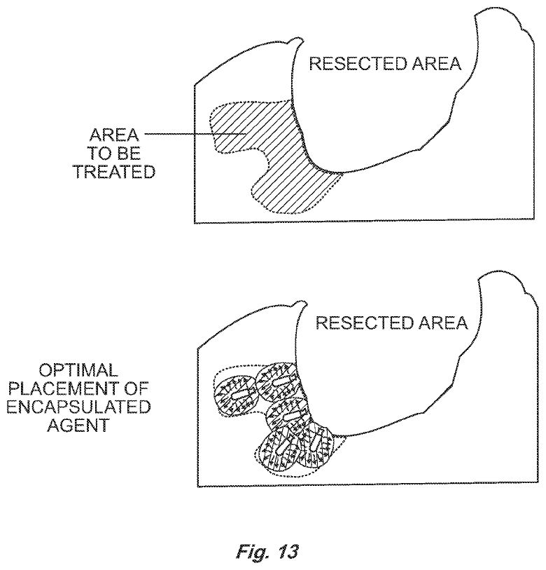

In some embodiments, the exterior shell has a circumferential opening for access to and from the interior. In such embodiments, the circumferential opening may be sealed by a temporary stopper. One non-limiting example of such an embodiment may be seen in FIG. 6B.

In some embodiments, the pharmaceutical composition comprises a therapeutic agent, a diagnostic agent, or a combination thereof. In some embodiments, the pharmaceutical composition comprises a plurality of therapeutic agents and/or diagnostic agents. In some embodiments, the pharmaceutical composition comprises a dye or other contrast agent. In some embodiments, the pharmaceutical composition comprises viruses or viral particles.

In some embodiments, a carrier device can contain between about 0.1 μL to about 100 μL of a solution of the therapeutic and/or diagnostic agents. In some such embodiments, the solution contains between about 1×10³ to about 1×10¹⁵ copies of a viral entity designed to penetrate the cells of surrounding living tissue.

In another aspect, provided herein are pharmaceutical composition delivery systems, the system comprising: a plurality of carrier devices described herein. In some embodiments, the system comprises at least 5 carrier devices. In some embodiments, the system comprises at least 10 carrier devices.

In still another aspect, provided herein are therapeutic injection systems, the system comprising: a cannula having a tissue-penetrating end and a payload-receiving end, the payload-receiving end configured to receive one or more carrier devices described herein.

In some embodiments, the payload receiving end is coupled to a feeder configured to sequentially feed carrier devices into the payload receiving end of the cannula.

In some embodiments, the tissue-penetrating end is positioned at a selected location within a tissue of a subject. In such embodiments, the tissue may be or may be proximate to at least one malignant neoplasm.

In some embodiments, the system further comprises a robot actuator to position the cannula.

In yet another aspect, provided herein are methods of providing a treatment plan for a subject in need thereof, the method comprising the steps of: (a) determining the spatial dimensions of a tissue or subtissue in need of treatment; (b) determining an optimal dose and time course for treatment; (c) inputting topological and medical data into a general-purpose computer pre-installed with software configured to determine recommended materials, dimensions, size, dosing, and/or spacing of a pharmaceutical composition delivery system as described herein; (d) the general-purpose computer determining and outputting a treatment plan comprising recommended materials, dimensions, size, dosing, and/or spacing for the pharmaceutical composition delivery system.

In some embodiments, the treatment plan comprises determining the number of carrier devices and where to position each carrier device in the tissue being treated. In some embodiments, the input data includes one or more MRI images of the tissue being treated. In some embodiments, the output includes a set of commands for a robotic system to control the therapeutic injection system. In some embodiments, the output includes a set of commands to a vision system and display to monitor manual positioning of the therapeutic injection system and indicate that the therapeutic injection systems is in an adequate position to place one or more of the carrier devices.

In still another aspect, provided herein are methods of treating a disease or condition in a tissue of a subject in need thereof, the method comprising: intraparenchymally positioning one or more carrier devices described herein into the tissue of the subject. In some embodiments of the method, two or more carrier devices are sequentially positioned by: (a) introducing a cannula into a first target location in the tissue, and positioning a first carrier device; (b) introducing the cannula into a second target location in the tissue, and positioning a second carrier device; (c) optionally repeating step (b) (for an nth target location in the tissue and positioning an nth carrier device).

In still another aspect, provided herein are methods of treating a malignant neoplasm in a brain of a subject in need thereof, the method comprising: intraparenchymally positioning one or more carrier devices described herein into brain tissue of the subject. In some embodiments of the method, two or more carrier devices are sequentially positioned by: (a) introducing a cannula into a first target location in the brain tissue, and positioning a first carrier device; (b) introducing the cannula into a second target location in the brain tissue, and positioning a second carrier device; (c) optionally repeating step (b) (for an nth target location in the brain tissue and positioning an nth carrier device).

In some embodiments of the method, the tissue may be a malignant neoplasm.

While certain features of the invention have been illustrated and described herein, many modifications, substitutions, changes, and equivalents will now occur to those of ordinary skill in the art. It is. therefore, to be understood that the appended claims are intended to cover all such modifications and changes as fall within the true spirit of the invention.

What is claimed:

1. A carrier device for intraparenchymal placement or implantation to release a pharmaceutical composition into biological tissue, comprising:
an exterior shell and an interior,
a matrix comprising the pharmaceutical composition, wherein the matrix is contained within the interior;
wherein the carrier device is configured to release the pharmaceutical composition upon intraparenchymal placement.

2. The carrier device of claim 1, wherein the exterior shell comprises a resorbable or biodegradable material.

3. The carrier device of claim 2, wherein the resorbable or biodegradable material comprises polylactic acid, polyglycolic acid, and/or poly (lactic-co-glycolic acid).

4. The carrier device of claim 1, wherein the carrier device has a maximum dimension of between about 500 μm and about 5 cm.

5. The carrier device of claim 1, wherein the carrier device has a substantially cylindrical shape.

6. The carrier device of claim 1, wherein the carrier device has a cross-sectional diameter ranging from between about 300 μm to about 5 mm.

7. The carrier device of claim 1, wherein the exterior shell comprises a bioresorbable hydrogel.

8. The carrier device of claim 1, wherein the exterior shell has a thickness of between about 5 μm to about 500 μm.

9. The carrier device of claim 1, wherein the matrix comprises a liquid, a powder, and or a solid.

10. The carrier device of claim 1, wherein the interior is substantially filled with the matrix comprising a solid or semisolid material in which a pharmaceutical agent is dissolved, suspended, or dispersed, and wherein the solid and semisolid material may be the same material or a different material as a material of the exterior shell.

11. The carrier device of claim 1, wherein the carrier device has an oblong shape.

12. The carrier device of claim 1, wherein the carrier device has a shape of: sphere, spheroid, hemisphere, hemispheroid, barrel, corpuscle, cylinder, ellipsoid, hemieillipsoid, lozenge, nephroid, teardrop, or vermiculate.

13. The carrier device of claim 11, wherein the oblong shape has a major axis dimension and a minor axis dimension, and wherein the major axis dimension may be determined by a user cutting the carrier device to length.

14. The carrier device of claim 1, wherein the exterior shell is disposed with one or more holes, each hole passing partly or fully through the exterior shell to the interior.

15. The carrier device of claim 14 wherein the holes pass fully through the exterior shell, each hole providing an opening through which the matrix may pass to and from the interior.

16. The carrier device of claim 14 wherein the holes are disposed in an annular band around an outer circumference of the exterior shell.

17. The carrier device of claim 14 wherein the holes are disposed on an isolated region of an outer surface of the exterior shell.

18. The carrier device of claim 14 wherein the holes are disposed substantially throughout the entirety of an outer surface of the exterior shell.

19. The carrier device of claim 1, wherein the exterior shell has a circumferential opening access to and from the interior.

20. The carrier device of claim 19, wherein the circumferential opening is sealed by a temporary stopper.

21. The carrier device according to any one of claims 14-18 wherein the holes have a diameter ranging from between about 100 nm to about 300 μm.

22. The carrier device according to any of claims 14-18 wherein the holes are plugged with opercula, the opercula having a thickness of between about 1 μm to about 300 μm, and the opercula comprising chitosan, Zein, and/or polyvinyl alcohol (PVA) or a combination thereof.

23. The carrier device of claim 1, wherein the matrix comprises a desiccatable hydrogel, which is dehydrated and shrunk, and which rehydrates and swells upon intraparenchymal placement.

24. The carrier device of claim 1, wherein the pharmaceutical composition comprises a therapeutic agent, a diagnostic agent, or combination thereof.

25. A pharmaceutical composition delivery system comprising a plurality of carrier devices according to any one of claims 1-24.

26. The pharmaceutical composition delivery system of claim 25 comprising at least 5 carrier devices.

27. The pharmaceutical composition delivery system of claim 26 comprising at least 10 carrier devices.

28. A therapeutic injection system comprising a cannula, having a tissue-penetrating end and a payload-receiving end, the payload-receiving end configured to receive one or more of carrier devices according to any one of claims 1-24.

29. The therapeutic injection system of claim 28, wherein the payload receiving end is coupled to a feeder configured to sequentially feed carrier devices into the payload receiving end of the cannula.

30. The therapeutic injection system of claim 28, wherein the tissue-penetrating end is positioned at a selected location within tissue.

31. The therapeutic injection system of claim 28, further comprising a robot actuator to position the cannula.

32. The therapeutic injection system of claim 30, wherein the tissue is or is proximate to a malignant neoplasm.

33. A method of providing a treatment plan for a subject in need thereof, the method comprising:
(a) determining the spatial dimensions of a tissue or subtissue in need of treatment;
(b) determining an optimal dose and time course for treatment;
(c) inputting topological and medical data into a general-purpose computer pre-installed with software configured to determine recommended materials, dimensions, size, dosing, and/or spacing of a pharmaceutical composition delivery system of claim 25; and
(d) the general-purpose computer determining and outputting a treatment plan comprising recommended materials, dimensions, size, dosing, and/or spacing for the pharmaceutical composition delivery system.

34. A method of treating a disease condition in a tissue of a subject in need thereof, the method comprising:
intraparenchymally positioning one or more carrier devices according to any one of claims 1-24 into the tissue of the subject.

35. The method of treating a disease condition of claim 34, wherein two or more carrier devices are sequentially positioned by
(a) introducing a cannula into a first target location in the tissue, and positioning a first carrier device,
(b) introducing the cannula into a second target location in the tissue, and positioning a second carrier device, and
(c) optionally repeating step (b).

36. The method of treating a disease condition of claim 34 wherein the tissue is a malignant neoplasm.

37. A method of treating a malignant neoplasm in a brain of a subject in need thereof, the method comprising:
intraparenchymally positioning one or more carrier devices according to any one of claims 1-24 into brain tissue of the subject.

38. The method of treating the malignant neoplasm of claim 37, wherein two or more carrier devices are sequentially positioned by
(a) introducing a cannula into a first target location in the brain tissue, and positioning a first carrier device,
(b) introducing the cannula into a second target location in the brain tissue, and positioning a second carrier device, and
(c) optionally repeating step (b).

39. The method of treating the malignant neoplasm of claim 37, wherein the method is performed on neoplastic tissue remaining after an initial resection the malignant neoplasm.

40. The method of treating the malignant neoplasm of claim 37, wherein the method is performed upon a recurrence of the malignant neoplasm.
